(19) United States
(12) Patent Application Publication
(10) Pub. No.: US 2008/0125513 A1
(43) Pub. Date: May 29, 2008

(54) Hydrophilic, Water-Swellable, Cross-Linked Matrix Having Incorporated Therein an Anti-Microbial Polymer

(76) Inventor: Soeren Kristiansen, Hundested (DK)

Correspondence Address: JACOBSON HOLMAN PLLC, 400 SEVENTH STREET N.W., SUITE 600, WASHINGTON, DC, 20004, US

(21) Appl. No.: 11/631,339

(22) PCT Filed: Jun. 27, 2005
(86) PCT No.: PCT/DK05/00431

§ 371 Date: Nov. 12, 2007

(30) Foreign Application Priority Data
Jun. 30, 2004 (DK) PA200401033

(51) Int. Cl. A61L 27/34

(52) U.S. Cl. 523/113; 525/55

(57) ABSTRACT

A hydrophilic, water-swellable, cross-linked matrix of a hydrophilic polymer having incorporated therein an anti-microbial polymer, said anti-microbial polymer carrying pendant groups providing the anti-microbial effect, said pendant groups being selected from primary amino groups, secondary amino groups, tertiary amino groups, quaternary ammonium groups, imino groups, and phosphonium groups.

### FIELD OF THE INVENTION

The present invention relates to a hydrophilic, water-swellable, cross-linked matrix of a hydrophilic polymer having incorporated therein an anti-microbial polymer. The present invention also relates to objects having a layer of such hydrophilic matrices, in particular medical devices.

### BACKGROUND OF THE INVENTION

WO 02/17725 (D1) and WO 02/28927 (D2) disclose anti-microbial oligomers as well as polymer formulations comprising such oligomers combined with water-insoluble polymers. The oligomers and polymer formulations are used as coatings, varnishes and paints on a wide range of products, e.g. products within the field of medicine, e.g. coatings on contact lenses, catheters, etc.

WO 00/69264 (D3), WO 00/69925 (D4), WO 00/69933 (D5), and WO 00/69935 (D6) disclose anti-microbial polymers for producing anti-microbial polymer surfaces. The polymers are used as coatings on, or are grafted onto, a wide range of products, e.g. products within the field of medicine, e.g. coatings on contact lenses, catheters, etc. brief description of the invention

D1-D6individually represent close prior art. The anti-microbial properties provided by the techniques disclosed in D1-D3 are established by coating an object, or grafting onto the surface of an object, an anti-microbial polymer or oligomer.

The problem underlying the present invention is to provide anti-microbial properties to hydrophilic, water-swellable, cross-linked matrices, in particular those useful in or in combination with medical devices such as catheters and wound dressings, without imparting the beneficial hydrophilic properties of said matrices.

Thus, the present invention provides an elegant solution to the above problem, cf. claim 1.

The invention further provides an object, cf. claim 8.

### DESCRIPTION OF THE INVENTION

### The Hydrophilic Matrix

As mentioned above, the present invention relates to a hydrophilic, water-swellable, cross-linked matrix of a hydrophilic polymer having incorporated therein an anti-microbial polymer, said anti-microbial polymer carrying pendant groups providing the anti-microbial effect, said pendant groups being selected from primary amino groups, secondary amino groups, tertiary amino groups, quaternary ammonium groups, imino groups, and phosphonium groups.

### Anti-Microbial Polymer

An important feature of the invention is the anti-microbial polymer which is incorporated in the hydrophilic matrix. The anti-microbial polymer is one which is carrying pendant groups providing the anti-microbial effect, said pendant groups being selected from primary amino groups, secondary amino groups, tertiary amino groups, quaternary ammonium groups, imino groups, and phosphonium groups. It should—of course—be understood that two or more such polymer may be used in combination.

By the term “pendant groups” is meant that the groups in question are covalently bound to the polymer backbone, but do not form part of the polymer backbone. Such groups may also be end-groups of a polymer chain, but are not exclusively such end-groups.

The anti-microbial polymer typically has a weight average molecular weight in the range of 2,000-500,000, such as 4,000-250,000 or 4,000-50,000 or 50,000-250,000.

Examples of polymers carrying primary amino groups (—NH₂) are those incorporating monomers selected from the group consisting of 1-amino-propen, methacrylic acid-2-aminoethylester-hydrochloride, acrylic acid-3-aminopropyl ester, aminopropylmethacrylamide, aminoethylvinyl ether, and 3-aminopropylvinyl ether.

Secondary amino groups may be of the type (—NHR¹) where R¹ is selected from the group consisting of C₁₋₆-alkyl, aryl, aryl-C₁₋₆-alkyl (such as benzyl), heteroaryl, and heteroaryl-C₁₋₆-alkyl (such as heteroaryl-methyl). Examples of polymers carrying secondary amino groups (—NHR¹) are those incorporating monomers selected from the group consisting of 1-(N-methyl)amino-propene, methacrylic acid-2-(N-methyl)aminoethylester-hydrochloride, acrylic acid-3-(N-methyl)aminopropyl ester, (N-methyl)aminopropylmethacrylamide, (N-methyl)aminoethylvinyl ether, and 3-(N-methyl)aminopropylvinyl ether.

Tertiary amino groups may be of the type (—N(R¹)₂) wherein each R¹ independently is selected as defined above. Examples of polymers carrying tertiary amino groups (—N(R¹)₂) are those incorporating monomers selected from the group consisting of 1-(N,N-dimethyl)amino-propene, methacrylic acid-2-(N,N-dimethyl)aminoethylester-hydrochloride, acrylic acid-3-(N,N-dimethyl)aminopropyl ester, (N,N-dimethyl)aminopropylmethacrylamide, (N,N-dimethyl)aminoethylvinyl ether, and 3-(N,N-dimethyl)aminopropylvinyl ether.

Quaternary ammonium groups may be of the type (—N⁺(R¹)₃) wherein each R¹ independently is selected as defined above. Examples of polymers carrying quaternary ammonium groups (—N⁺(R¹)₃) are those incorporating monomers corresponding to those defined above for tertiary amines. For this series, the counter-ion (the anion) to the ammonium group may any conventionally used counter-ion, or may be selected from anions which in themselves provides an anti-microbial effect.

Imino groups may be of the type (—N═C(R²)₂) wherein each R² independently is selected from hydrogen and the groups defined above for R¹. Examples of polymers carrying imino groups (—N═(R²)₂) are those incorporating monomers corresponding to those defined above for primary amines condensed with aldehydes or ketones.

Phosphonium groups may be of the type (—P⁺(R³)₃) wherein R³ is selected as R¹ above. Examples of polymers carrying phosphonium groups (—P⁺(R³)₃) are those incorporating monomers selected from the group consisting of trimethylphosphoniumethylacrylate and trimethylphosphonlumethylmethacrylate. In this series, the counter-ion to the phosphonium groups may be selected as defined above for the ammonium group.

In a preferred embodiment, the anti-microbial polymer includes units derived from monomers selected from the group consisting of methacrylic acid-2-tert-butylaminoethylester, methacrylic acid-2-diethylaminoethylester, methacrylic acid-2-diethylaminomethylester, acrylic acid-2-tert.-butylaminoethylester, acrylic acid-3-dimethylaminopropylester, acrylic acid-2-dlethylaminoethylester, acrylic acid-2-dlmethylaminoethylester, dimethylaminopropylmethacrylamide, diethylamino-propylmethacrylamide, acrylic acid-3-dimethylaminopropylamide, 2-methacryloyloxyethyltrimethylammonium methosulphate, methacrylic acid-2-diethylaminoethylester, 2-methacryloyloxyethyltrimethylammonium chloride, 3-methacryloylaminopropyltrimethylammonium chloride, 2-methacryloyloxyethyltrimethylammonium chloride, 2-acryloyloxyethyl-4-benzoyldimethylammonium bromide, 2-methacryloyloxyethyl-4-benzoyldimethylammonium bromide, allyltriphenylphosphonium bromide, allyltriphenylphosphonium chloride, 2-acrylamido-2-methyl-1-propanesulfonic acid, 2-diethylaminoethylvinylether and 3-aminopropylvinylether.

Other monomers may be used as the balance in such anti-microbial polymers. Examples hereof are acrylates and methacrylates, e.g. acrylic acid, methyl acrylate, methacrylic acid, methyl methacrylic acid, ethyl methacrylate, ethyl acrylate, propyl methacrylate, propyl acrylate, isopropyl methacrylate, isopropyl acrylate, tert-butyl methacrylate, tert-butyl acrylate, butyl methacrylate, butyl acrylate, etc.

The anti-microbial polymer is preferably selected from the group consisting of polymethacrylates and polyacrylates.

It is preferred that at least 10%, such as at least 20%, of the units of the anti-microbial polymer are units carrying the pendant groups providing (or at least contributing to) the anti-microbial effect.

The content of the anti-microbial polymer is typically up to 50%, e.g. 0.1-20% based on the total weight of the matrix (including the anti-microbial polymer).

More particular examples of anti-microbial polymers are those produced by polymerisation of one of several monomers as disclosed in patents DE 199 21 894 A1, DE 199 21 897 A1, DE 199 21 898 A1, DE 199 21 900 A1and WO 02/17725 A1 or equivalent, and commercial products such as AMINA® T 100 (Degussa).

In a preferred embodiment of the invention, the anti-microbial polymer does not contain vinylic unsaturated groups.

The anti-microbial polymer may be introduced to the hydrophilic matrices by different approaches but preferably through direct formulation into the final mixture of the matrix, as will be described in detail further below. In one intriguing embodiment, the anti-microbial polymer chains are grafted on the chains of the hydrophilic polymer(s), or vice versa. Alternatively, the hydrophilic polymer(s) and the anti-microbial polymer(s) are reacted so as to form block copolymers.

In one embodiment of the invention, the antimicrobial polymer is not covalently bound to the hydrophilic, water-swellable, cross-linked matrix of a hydrophilic polymer. Such polymer systems may be obtained by equilibrating the hydrophilic, water-swellable matrix after it has been cross-linked as described below. Such polymer systems may also be achieved by appropriate selection of the curing conditions and the selection of the cross-linker.

### Water-Swellable Cross-Linked Hydrophilic Matrix

The most fundamental constituent of the water-swellable, cross-linked hydrophilic matrix is the hydrophilic polymer.

In the present context, the term “water-swellable” means that the dry matrix including the anti-microbial polymer is able to absorb more than 30% of its weight in pure water. However, preferably the dry matrix shall be able to absorb at least 100%, such as at least 200%, e.g. at least 400%, or at least 600%, or even at least 1000%, of its weight in pure water.

Thus, in one embodiment, the weight ratio between the non-swollen (dehydrated) and the instantly swollen (hydrated) matrix will be at least 1:1.3, e.g. at least 1:2. “Instantly swollen” means swollen by soaking in water for 90 seconds.

In some further interesting embodiments, the ratio of the thickness of the hydrophilic matrix in water swollen form (h_{water}) to the thickness of the hydrophilic matrix in dry form (h_{dry}) is at least 2:1, such as at least 3:1. The thickness of the matrix in dry form (h_{dry}) is typically at least 1 μm.

By the term “cross-linked” is meant that the hydrophilic matrix is covalently cross-linked to such an extent that it is insoluble in water, i.e. that the integrity of the matrix will be preserved even after prolonged soaking in water. The term “insoluble” means that the hydrophilic matrix does not disintegrate upon soaking in water, i.e. at least such that it has not fully disintegrated upon storage for 24 hours at 20° C. In 1 L of pure water per gram matrix.

The matrix typically comprises at least one water-swellable hydrophilic polymer which provides the water-swellability. In most instances, the matrix comprises at least 50% by solids weight of hydrophilic polymers, such as at least 80% or 90% or more by solids weight of water-swellable hydrophilic polymers. The balance of the matrix is constituted by the anti-microbial polymer and any optional other constituents.

The term “hydrophilic polymer” means in this context any polymeric compound which (in non-crosslinked form) is soluble in water in concentrations higher than 100 g/L.

Examples of water-swellable hydrophilic polymers are those selected from polyvinylpyrrolidone, polyvinyl alcohol, polyacrylic acid, polymethacrylic acid, polyacrylic amides, polymethacryl amides, and grafts or copolymers of any of these polymers, e.g. copolymers with maleic anhydride (such as poly(methyl vinyl ether/maleic anhydride)), succinic anhydride or the corresponding acids, as well as polyamides, polyethylene glycols (PEG), gelatine, polysaccharides (e.g. cellulose derivatives such as carboxymethylcellulose, cellulose acetate, and cellulose acetate propionate, and chitosan), hydrophilic polyurethanes (e.g. one-shot or prepolymer-based polyurethanes), and carboxylated butadiene styrene rubbers. Preferably, the hydrophilic matrix comprises at least one of the before-mentioned hydrophilic polymers.

In a first main embodiment, the matrix is in the form of a hydrophilic coating on a substrate.

In this first main embodiment, it is preferred that the hydrophilic matrix comprises at least one hydrophilic polymer selected from the group consisting of polyvinylpyrrolidone, polyvinyl alcohol, polyacrylic acid, polymethacrylic acid, polyacrylic amides, polymethacryl amides, and grafts or copolymers of any of these polymers, e.g. copolymers with maleic anhydride (such as poly(methyl vinyl ether/maleic anhydride)), succinic anhydride or the corresponding acids, as well as polyethylene glycols (PEG) polysaccharides (e.g. carboxymethylcellulose, cellulose acetate, cellulose acetate propionate, chitosan), and hydrophilic polyurethanes (e.g. one-shot or prepolymer-based polyurethanes).

Most preferably, the hydrophilic polymer of the coating is selected from the group consisting of polyethylene glycol, polyvinylpyrrolidone and copolymers including polyvinylpyrrolidone, e.g. polyvinylpyrrolidone-vinyl acetate copolymers. When using the pure polyvinylpyrrolidone (poly(N-vinyl-2-pyrrolidone); PVP), various chain lengths may be selected each giving various characteristics to the coating. Typically, such polyvinylpyrrolidone polymers have a number average molecular weight of above 100,000. As an example, PVP K-90 with a molecular weight of 1,200,000 can be selected but other types of PVP with molecular weights In the range of 100,000-3,000,000 may advantageously be used, e.g. PVP K-120 or PVP K-45.

Other radiation curing hydrophilic polymers comprising unsaturated vinylic double bonds can also suitably be used for the coating. Such polymers may be made by copolymerising into a prepolymer an acrylic substance like dimethylaminoethylmethacrylate with N-vinylpyrrolidone, methacrylic acid, methacrylic esters, methyl vinyl ether etc. Such a prepolymer is typically coated to the surface and ultimately radiation cured. The hydrophilic polymer of the coating may further be made by adding monomers of acrylic nature to the above-mentioned types of polymers. Polyethylene glycols and polyvinylpyrrolidone are particularly useful for such hydrophilic coatings.

In one embodiment, the radiation curing hydrophilic polymer is a saturated polymer.

In one interesting embodiment, the matrix comprises at least 50% by solids weight of one or more hydrophilic polymers selected from the group consisting of polyethylene glycol, polyvinylpyrrolidone and copolymers including polyvinylpyrrolidone, e.g. polyvinylpyrrolidonevinyl acetate copolymers. These types of polymers are very useful for cross-linking by radiation.

Furthermore, the weight ratio between the non-swollen (dehydrated) and the instantly swollen (hydrated) coating will typically be at least 1:4.

In a second main embodiment, the matrix is in the form of a hydrogel.

In this second main embodiment, it is preferred that the water-swellable hydrophilic matrix comprises at least one hydrophilic polymer selected from the group consisting of polyvinylpyrrolidone, polyvinyl alcohol, polyacrylic acid, polymethacrylic acid, polyacrylic amides, polymethacrylic amides, and copolymers thereof (e.g. poly(methyl vinyl ether/maleic anhydride)), as well as polyamides, polyethyleneglycols (PEG), polysaccharides (e.g. cellulose derivatives such as carboxymethylcellulose, cellulose acetate, and cellulose acetate propionate, and chitosan), and hydrophilic polyurethanes (e.g. one-shot or prepolymer-based polyurethanes).

Most preferably, the matrix comprises at least 50% by solids weight of the total amount of polymers of one or more hydrophilic polymers selected from the group consisting of polyethylene glycol, polyvinylpyrrolidone and copolymers including polyvinylpyrrolidone.

Furthermore, the weight ratio between the non-swollen (dehydrated) and the swollen (hydrated) hydrogel will typically be at least 1:4. It should be noted that the hydrogel typically is sized and packed in swollen (hydrated) form.

In a third main embodiment, the matrix is in the form of a hydrophilic foam.

In this third main embodiment, it is preferred that the water-swellable hydrophilic matrix comprises at least one hydrophilic polymer selected from the group consisting of polyvinylpyrrolidone, polyvinyl alcohol, polyacrylic acid, polymethacrylic acid, polyacrylic amides, polymethacrylic amides, and copolymers thereof, as well as gelatine, polysaccharides (e.g. carboxymethylcellulose, cellulose acetate, cellulose acetate propionate, chitosan), hydrophilic polyurethanes (e.g. one-shot or prepolymer-based polyurethanes), and carboxylated butadiene styrene rubbers.

Examples of useful hydrophilic polyurethanes are those prepared from hydrophilic isocyanate terminated polyether prepolymers (e.g. prepolymers of polyethylene oxide) and water. Commercial examples include those known as Hypol foams, e.g. those prepared from Hypol hydrophilic prepolymers marketed by W.R. Grace and Co, such as Hypol BJ, Hypol JI, Hypol JM, and Hypol JT.

Most preferably, the matrix comprises at least 50% by polymer solids weight of one or more hydrophilic polymers selected from the group consisting of hydrophilic polyurethanes, polyacrylates, polyvinyl alcohol, and polyvinylpyrrolidone, in particular hydrophilic polyurethanes.

Furthermore, the weight ratio between the non-swollen (dehydrated) and the instantly swollen (hydrated) hydrophilic foam will typically be at least 1:4. With respect to the foam bulk as a whole (e.g. including the pores of the foam), ratio between the non-swollen bulk and the hydrated bulk holding water is at least 1:4, such as at least 1:5.

In order to obtain an even release profile for the matrix (e.g. coating, hydrogel or foam) of the anti-microbial polymer and the hydrophilic polymer, the hydrophilic polymer and the anti-microbial polymer preferably form a homogeneous water-swellable coating. This can be achieved by simple mixing of the polymer constituents and/or precursors upon establishment of the matrix.

In one embodiment, the anti-microbial polymer is covalently linked to the hydrophilic polymer, e.g. by grafting or by graft polymerization. This being said, it is still important that grafting not only occur on the surface of the matrix, but that the anti-microbial polymer truly is incorporated in the matrix. Thus, grafting is preferably performed before formation of the cross-linked matrix.

The present invention is particularly relevant for fairly thick coatings when swollen as those useful for medical devices, such as catheters, e.g. urinary catheters. Thus, in the first main embodiment (a coating), the thickness in dry form (h_{dry}) of the hydrophilic coating is typically at least 1 μm, or at least 3 μm, such as 6 μm, e.g. 3-500 μm, such as 6-150 μm. In the second main embodiment (a hydrogel), the thickness in dehydrated form (h_{dry}) of the hydrogel is typically at least 10 μm, such as at least 20 μm, e.g. 10-5000 μm, such as 20-400 μm. In the third main embodiment (a foam), the thickness in dry form (h_{dry}) of the hydrophilic foam bulk is typically at least 100 μm, such as at least 200 μm, e.g. 100-10,000 μm, such as 500-5000 μm.

### The Object

The present invention also provides an object comprising a substrate material and a layer covering at least a part of the surface of said substrate material, said layer consisting of a hydrophilic, water-swellable, cross-linked matrix as defined herein.

### Substrate

The hydrophilic matrix may be combined with a substrate so as to provide hydrophilic as well as anti-microbial properties to an object. Such objects, e.g. medical devices and medical device elements, can be formed from a variety of types of basic materials, such as plastics, metals, glasses, ceramics, etc. Typical examples of plastic materials for medical devices are polymers such as polyurethanes and copolymers thereof, or polyether block amides such as Pebax™ or other polymer materials including polyvinyl chloride, polyamide, silicone, styrene-ethylene/butylene-styrene block copolymers (SEBS), styrene-isoprene-styrene block copolymers (SIS), styrene-ethylene/propylene-styrene block copolymers (SEPS), ethylene-vinyl acetate copolymers (EVA), polyethylene (PE), metallocene-catalyzed polyethylene, and copolymers of ethylene and propylene or mixtures of such. For some of the combinations of substrate polymers and hydrophilic matrices (e.g. polyethylene as a substrate polymer and PVP as the hydrophilic polymer) a primer coat (e.g. a PVC or polyurethane solution) may advantageously be applied before application of the polymer solution. The polymeric substrate will normally be inherently hydrophobic. Still further the substrate may be moulds, films or meshes of metal, glass or equivalent. Currently very relevant materials for medical devices are polyurethanes and copolymers thereof.

Within the present context, the medical device has on at least a part of the surface thereof (i.e. on at least a part of the surface of the basic material) a layer of a hydrophilic matrix as defined herein. In some embodiments, the hydrophilic layer is established on the full (outer) surface of the substrate polymer, and in some other embodiments, only to a part of the surface. In the most relevant embodiments, the layer is applied to at least a part of the surface (preferably the whole surface) of a medical device that—upon proper use—comes into direct contact with body parts of the person for which the medical device is intended.

The substrate is preferably at least part of an object, e.g. a medical device. Thus, a further aspect of the present invention relates to an object comprising a substrate material and a layer covering at least a part of the surface of said substrate material, said layer consisting of a hydrophilic matrix as defined herein. As mentioned above, the substrate material is typically selected from the group consisting of plastics, metals, glass, and ceramics.

Preferably, the object is a medical device or a medical device element, preferably a medical device or medical device element selected from the group consisting of catheters, endoscopes, laryngoscopes, tubes for feeding, tubes for drainage, guide wires, condoms, urisheaths, barrier coatings, stents and other implants, extra corporeal blood conduits, membranes, blood filters, devices for circulatory assistance, dressings for wound care, urine bags and ostomy bags.

Currently very relevant medical devices or medical device elements are catheters and catheter elements. In these instances, the matrix is preferably in the form of a hydrophilic coating.

Other currently very relevant medical devices or medical device elements are dressings for wound care (wound dressings). In these instances, the matrix is typically in the form of hydrogel or a hydrophilic foam.

### Preparation of the Hydrophilic Matrix

It is important that the anti-microbial polymer is truly incorporated in the hydrophilic matrix (and not only present on the surface of the hydrophilic matrix!) so as to provide the beneficial effects, i.e. that the anti-microbial polymer is substantially homogeneously distributed in the matrix (e.g. coating, hydrogel or foam). Thus, a hydrophilic matrix defined herein may be prepared by one of the following general procedures.

### Hydrophilic Matrix in the Form of a Hydrophilic Coating on a Substrate

### (A) Preparation of Matrix with Anti-Microbial Polymer

In one variant, the anti-microbial polymer may be added to the polymer solution intended to produce the matrix on the medical device. In particular, the ingredients required for the formation of the matrix may be solubilized in water or any other aqueous mixture (e.g. ethanolic mixtures) or organic solvent. Currently preferred organic solvents include those selected from N-methyl-2-pyrrolidone, γ-butyrolactone, dimethyl sulfoxide, acetone, 1,3-dioxolane and dimethyl formamide. However, any solvent can in principle be used for the vehicle. Other options may therefore include methylethylketon, diethylether, dioxan, hexan, heptan, benzol, toluol, chloroform, dichlormethan, tetrahydrofuran and acetonitril, 1,3-dioxolane and other ethers, acetone and other ketones, dimethylsulfoxide and other sulfoxides, dimethyl formamide and other amides, N-methyl-2-pyrrolidone and other lactams, glycols, glycol ethers, glycol esters, other esters, amines, heterocyclic compounds, alkylated urea derivatives, liquid nitrites, nitroalkanes, haloalkanes, haloarenes, trialkyl phosphates, dialkyl alkanephosphonates, and other commonly known organic solvents. The preferred solvents may either be used singly or in combination. Typically, the solvent(s) constitute(s) 50-95 wt % of the polymer solution. In practical, the choice of mixture may be selected on the basis of the substrate since specific organic solvents may be required for the partly solubilization or swelling of the substrate surface. For example in a preferred embodiment of the invention, the vehicle (solvent(s), plasticizer(s), etc.) for the polymer may comprise mixtures of water, ethanol or ethanol and N-methyl-2-pyrrolidone.

Thus, in one embodiment of the invention the polymer solution used to produce the matrix may comprise:
10⁻⁵-20 wt % of the anti-microbial polymer,
0.2-50 wt % of a hydrophilic polymer,
0-10 wt %, such as 0.05-5 wt %, of a cross-linker,
0-50 wt % of one or more osmolality increasing agents, and
20-99.5 wt % of solvent(s) and/or plasticizer(s).

The polymer solution should preferably have a pH in the range of 3.5-8.5. The desirable pH value is typically obtained by addition of a buffer to the polymer solution.

The preferred plasticizers are glycols i.e. glycerol, polyethylene glycols, polypropylene glycols or acetyl triethyl citrate, dimethyl sulfone, ethylene carbonate, glycerol diacetate, glycerol triacetate, hexamethylphosphoramide, isophorone, methyl salicylate, N-acetyl morpholine, propylene carbonate, quinoline, sulfolane, triethyl citrate, and triethyl phosphate. Particular examples are acetyl triethyl citrate. The plasticizers may be used singly or in combination. The plasticizer(s) preferably constitute(s) 1-40 wt %, such as 3-30 wt %, of the polymer solution.

Examples of osmolality increasing agents are alkali metal (e.g. lithium, sodium, potassium, etc.) and earth alkali metal (magnesium, calcium, etc.) nitrates, alkali metal and earth alkali metal sulfates, alkali metal and earth alkali metal chlorides, glycine, glycerol and urea. An osmolality increasing agent is not strictly necessary, but it is often relevant in order to improve the comfort upon use if the coating is intended for a medical device. When present, the osmolality increasing agent(s) typically constitute(s) 1-80 wt %, e.g. 1-50 wt %, such as 2-30 wt %, of the polymer solution. One or more additives may be included in the polymer solution, e.g. so as to facilitate the cross-linking of the hydrophilic polymer or so as to improve bonding of the polymer to the substrate surface. Such additives are known in the art and may include UV initiators, e.g. as described in the applicant's earlier WO 98/58990.

The polymer solution comprising the above ingredients can be applied to the substrate surface by conventionally techniques (dipping, spraying, incubation, rolling etc.) and may subsequently be dried by evaporation of at least a part of the solvent(s) and/or plasticizer(s).

The matrix can then later be swollen with water or an aqueous solution (e.g. physiological saline) prior to use in order to give low friction properties.

Thus, an object with a hydrophilic coating according to the invention may be provided through a method comprising the steps of:
(I) providing a substrate polymer having the substrate surface;
(II) providing a polymer solution (e.g. as the one described above) comprising one or more hydrophilic polymers and an anti-microbial polymer;
(III) applying said polymer solution to said substrate;
(IV) optionally evaporating at least a part of the solvent(s) and/or plasticizer(s) from said polymer solution present on said substrate polymer surface; and
(V) curing said hydrophilic polymer.

### Step (I)

As mentioned above, the substrate may be the native surface of a medical device or another object, or may be surface treated so as to facilitate strong bonding of the hydrophilic matrix to the substrate. The surface of the substrate may be the complete physical surface or a fraction thereof. For instance with many medical devices, it is only necessary to coat the part of the substrate polymer surface, which comes into direct contact with the surface of living tissue when in use. Thus, the step of providing a substrate having the substrate surface will be evident for the person skilled in the art. In a special embodiment, the surface of the substrate can be in the form of a mould. After formation of the matrix, the matrix is removed from the mould and the mould may therefore not be an integrated part of the final medical device.

### Step (II)

The selection of polymer solution is crucial for the method of the invention. The choice of hydrophilic polymer and optionally monomers corresponding to the hydrophilic polymer, vehicle including solvent(s) and plasticizer(s) and additives including the anti-microbial oligomer or polymer is described above. The solution may be prepared by mixing the components with the hydrophilic polymer in order to obtain the polymer solution. The mixing order is not particularly critical as long as a homogeneous (and possibly clear) solution is obtained. Thus, the step of actual preparation of the polymer solution may be evident for the person skilled in the art in view of the above directions with respect to choice of actual components.

### Step (III)

Application of the polymer solution to said substrate surface is conducted following conventional methods such as bar coating, reverse roll coating, dip coating, spray coating, application by means of brushes, rollers, etc., as will be evident for the person skilled in the art. With due consideration of the production process, it is preferred that the application of the polymer to the substrate polymer surface is performed by dipping the medical device (or the relevant surface thereof) into the polymer solution when non-planar and using bar coating with or for planar species.

In a preferred embodiment for coatings, the polymer solution is applied to a substrate polymer surface in one single application step, such as in a one-dip process. It should however be understood that the substrate polymer may be primed in one or more preceding step and that such a preceding step may be applied in addition to a single application step (one-dip process).

### Step (IV)

After application of the polymer solution to the substrate surface, any organic solvent(s) and/or plasticizer(s) is/are evaporated from the polymer solution present on said substrate polymer surface. The solvents may be removed by passive evaporation, by leading a stream of air over the surface of the substrate, or by applying a reduced pressure over the surface of the substrate. Furthermore, it may be necessary or desirable to increase the temperature of the substrate or the air surrounding the substrate to speed up the evaporation process. Preferably, the evaporation process is facilitated by drying the substrate with the polymer solution at a temperature of between 25-100° C. depending on the thermostability of the substrate and the polymer. Typically, the substrate (e.g. a medical device) is dried in an oven. When water constitutes the entire solvent system or a part of the solvent system, preferably no or only part of the water is evaporated, because a subsequent rehydration step can thereby be eliminated. This particularly applies for hydrogels (hydrogels).

### Step (V)

Although the curing of the hydrophilic polymer of the polymer solution may be effected during the optional drying process (step (iv)), it is often desirable to specifically induce curing (cross-linking) of the hydrophilic polymer. Most advantageously, the free-radical curing (and cross-linking) is performed by application of radiation, e.g. UV radiation, beta- or gamma radiation. The method of curing, in particular the frequency and intensity of the UV light, is depending on the choice of photoinitiator (i.e. the cross-linking agent). The person skilled in the art will know the means and procedures necessary for efficient curing, e.g. also how to conduct the curing by other means, e.g., by thermocuring involving labile groups of the polymer(s).

In a preferred embodiment as for cross-linked coatings of the above method, the hydrophilic coating is prepared by dipping a medical device having a substrate polymer surface of polyurethane in a solution of the preferred hydrophilic polymer (i.e. polyvinylpyrrolidone), a photoinitiator, one or more plasticizers. The device is subsequently dried in an oven at a temperature of 25-100° C. so as to remove a substantial portion of the solvent and radiated with specific ultraviolet light to effect cross-linking.

### (B) Equilibration of Existing Hydrophilic Coating

In another variant of providing the hydrophilic coating of the invention is to dissolve the anti-microbial polymer in a solvent, and equilibrate an already existing hydrophilic matrix with this solution so as to allow the anti-microbial polymer to penetrate the hydrophilic matrix. The hydrophilic coating is, thus, essentially prepared as described above.

Thus, in one variant, the aqueous solution used to equilibrate the matrix may comprise:
10⁻⁵-20 wt % of the anti-microbial polymer,
0-10 wt % of polyvinylpyrrolidone and/or of polyethyleneglycol,
0-50 wt % of one or more osmolality increasing agents, and
20-99.5 wt % of solvent(s) and/or plasticizer(s).

The aqueous solution should preferably have a pH in the range of 3.5-8.5. The desirable pH value is typically obtained by addition of a buffer to the polymer solution.

Thus, an object with a hydrophilic coating according to the invention may be provided through a method comprising the steps of:
(I) providing a substrate polymer having the substrate surface;
(II) providing a polymer solution (e.g. as the one described above) comprising one or more hydrophilic polymers;
(III) applying said polymer solution to said substrate;
(IV) optionally evaporating at least a part of the solvent(s) and/or plasticizer(s) from said polymer solution present on said substrate polymer surface;
(V) optionally curing said hydrophilic polymer;
(VI) adding a solution containing the anti-microbial polymer to the hydrophilic matrix; and
(VII) curing said hydrophilic polymer, if not already cured in step (V).

The steps (I)-(V) and (VII) are described further below. Step (VI) can be effected by soaking the hydrophilic coating in the solvent defined above for a sufficient period of time. Typically, the concentration of the anti-microbial polymer is in the range of 1 mg/L to 10 g/L.

An advantage of this method is that the step (VI) of allowing the anti-microbial polymer to penetrate the hydrophilic matrix does not necessitate any changes in the procedure for establishing the hydrophilic matrix as such (see above under (A))

It should be noted that the hydrophilic matrix may be cross-linked either before equilibration with the anti-microbial polymer, or after equilibration with the anti-microbial polymer.

If the cross-linking (curing) is effected before equilibration, any cross-linking between chains of the hydrophilic polymer and chains of the anti-microbial polymer can be avoided, whereby the anti-microbial polymer will be able to penetrate into and be released from the cross-linked hydrophilic matrix.

If the cross-linking (curing) is effected after equilibration, the curing conditions, including selection of cross-linker, can be selected such that cross-linking between chains of the hydrophilic polymer and chains of the anti-microbial polymer can be obtained if desirable. In any event it is believed that the equilibration of the (non-cross-linked) hydrophilic matrix with the aqueous solution of the anti-microbial agent before curing (cross-linking) can have a positive effect on the friction force and the water drain-off time of the final coating, e.g. as it is described in the applicant's earlier European patent No. 1 131 112 B1.

### (C) Combination

Alternatively, in a third embodiment both approaches may be combined; the anti-microbial polymer may be present in the solution required to equilibrate the matrix and be present in the polymer solution required for the production of the hydrophilic matrix.

Thus, an object with a hydrophilic coating according to the invention may be provided through a method comprising the steps of:
(I) providing a substrate polymer (e.g. as the ones described above) having the substrate surface;
(II) providing a polymer solution comprising one or more hydrophilic polymers and an antimicrobial polymer;
(III) applying said polymer solution to said substrate;
(IV) optionally evaporating at least a part of the solvent(s) and/or plasticizer(s) from said polymer solution present on said substrate polymer surface;
(V) optionally, but preferably, curing said hydrophilic polymer;
(VI) adding a solution containing the anti-microbial polymer to the hydrophilic matrix; and
(VII) curing said hydrophilic polymer, if not already cured in step (V).

### Hydrophilic Matrix in the Form of a Hydrogel

A hydrogel may in principle be prepared essentially as described above for hydrophilic coating, however with the difference that the hydrogel is prepared on a pseudo-substrate, namely a substrate (e.g. a mould, film or a sheet of silicone- or paraffin-coated paper) from which the hydrogen will be separated before use, or on a film.

In some aspects, the procedure may be simplified compared with the procedure for preparing the coatings (see above) in that the “solvent” typically is water whereby any drying steps conducted with the purpose of removing organic solvent may be eliminated.

Thus, in one embodiment of the invention the polymer solution used to produce the matrix (hydrogel) may comprise:
10⁻⁵-20 wt % of the anti-microbial polymer,
0.2-50 wt % of a hydrophilic polymer,
0-10 wt %, such as 0.05-5 wt %, of a cross-linker,
0-50 wt % of one or more osmolality increasing agents, and
10-99.5 wt % of water or an aqueous solution.

The polymer solution should preferably have a pH in the range of 3.5-8.5. The desirable pH value is typically obtained by addition of a buffer to the polymer solution.

Thus, the hydrogel according to the invention may be provided through a method comprising the steps of:
(I) providing a pseudo-substrate or a film having a surface;
(II) providing a polymer solution (e.g. as the one described above) comprising one or more hydrophilic polymers and an anti-microbial polymer;
(III) applying said polymer solution to said substrate; and
(IV) curing said hydrophilic polymer.

### Hydrophilic Matrix in the Form of a Hydrophilic Foam

Hydrophilic polyurethanes represent a suitable example of hydrophilic foams. One typical type of preparation of such foams is known from the literature, see, e.g., “Hypol: Foamable hydrophilic polymers—laboratory procedures and form formulations”, a booklet published by W.R. Grace and Co.; and GB 1 429 711 B or GB 1 507 232 B. Alternatively, the hydrophilic foam may be prepared by foaming and freeze-drying other hydrophilic polymers, e.g., polyvinyl alcohol, gelatine, etc., and subsequently cross-linking the polymer by heating or by chemical activation. Alginate may be cross-linked by formation of calcium ion cluster-like cross-links.

Thus, the hydrophilic foam according to the invention may be provided through a method comprising the steps of:
(I) mixing the foam raw materials with the anti-microbial polymer;
(II) placing the reaction mixture in a desired geometry and allowing for reaction; and
(III) post-curing the foam and optionally drying the hydrophilic foam.

Alternatively, the method comprises the steps of:
(I) providing a hydrophilic foam;
(II) impregnating the hydrophilic foam with solution of an anti-microbial polymer; and
(III) drying the impregnated hydrophilic foam.

### After-Treatment

The hydrophilic matrix (coating, hydrogel or foam) may afterwards be dried or may be sized and packed in a suitable form. For medical devices, the package is preferably sterilized subsequent to packing.

The hydrophilic matrix, e.g. In form of a coating or a hydrogel, may become highly lubricious when sufficiently wet as the coating takes up a significant amount of water, which leaves a non-bonded layer of free water molecules at the surface of the coating. The non-bonding character of the surface water is believed to cause the low friction of the wet coating. Hence, the coating when applied to a biomedical or other device will improve biocompatibility and patient compliance. However, for most applications there will be high demands to the internal and the bonding strength for the coating. Thus, in a preferred embodiment of the invention the coating on the medical device may be packed in an un-swollen—or dry—condition, and saturated with a solution preferentially water before use. The solution used to saturate the coating may be a part of the packing device and therefore provided by the manufacturer, or the solution may be added and provided by the end-user. Alternatively, the coating on the medical device may also be packed in a swollen state and the solution is therefore added by the manufacturer prior to the sterilization process.

### EXAMPLES

All quantities indicated herein as “parts” refer to parts by weight.

### Materials

PVP K-90 (molecular weight 1.2×10⁶ g/mol) was obtained from ISP Technologies.
The UV catalyst Esacure® KIP 150 was obtained from Lamberti SpA.
N-methyl-2-pyrrolidone was obtained from Merck.
Triethyl citrate (Citrofol Al) was obtained from Jungbunzlauer.
Poly-(2-tert-butylaminoethyl methacrylate) was obtained from Degussa AG.
AMINA® T 100 was obtained from Degussa AG.

### Example 1

### Hydrophilic Coating

6 parts polyvinylpyrrolidone (PVP) K-90, optionally 1 part poly-(2-tert-butylaminoethyl methacrylate) and 0.03 parts Esacure® KIP 150 was dissolved in 77.97 parts ethanol, 5 parts N-methyl-2-pyrrolidone (NMP), and 10 parts triethylcitrate, and magnetically stirred overnight. The coating was applied to polyurethane catheters, and the catheters were incubated for 30 min at 70° C. before UV cured. The catheter coating were either packed in aluminium foil in a dry state or with a solution containing the antimicrobial polymer. The catheters were β-sterilized and stored for 12 days at 4° C., at room temperature or at 60° C.

In brief, the coatings we prepared as follows:
1) a control coating without the antimicrobial polymer
2) a coating prepared with the antimicrobial polymer
   both sterilized and stored dry and swollen for 90 s prior to the antimicrobial test.
3) a coating prepared as in 2) but sterilized and stored with 10 ml of saline water containing 5% PVP.
4) a coating prepared as in 1) but sterilized and stored with 10 ml of saline water containing 5% PVP and the antimicrobial polymer.

One part coating from coating 1-4) were dissolved in 3 part of saline water containing approximately 10⁹ colony forming units (CFU) bacteria per ml. Samples were harvested after 2 hours, diluted approximately 10⁶ fold in saline water, and plated onto sterile agar plates.

### Example 2

### Hydrophilic Coating

The antimicrobial polymer was labelled with ³H-proline by the use of the coupling agent EDC (Pierce) at pH 9.5 and added in trace amount to a solution as prepared as indicated in example 1. The concentration of the labelled antimicrobial polymer in the hydrophilic coating and in the surround solution (medium) was evaluated at various time points. As shown below, the antimicrobial hydropfobic polymer is released from the hydrophilic coating into the surround medium until a steady state condition is achieved after approximately 90 min.

### Example 3

### Hydrophilic Coating

A vehicle solution was prepared by solubilizing the antimicrobial polymer in saline water containing 5% PVP by the addition of HCl. The solution was diluted with the same solution without the antimicrobial polymer. Each solution was used to equilibrate a matrix. The antimicrobial potency of each matrix was tested by incubating the matrix together with a bacterial test solution. The bacteria and the pre-swollen matrix were incubated for 5 min, before samples were collected, diluted and transferred to agar plates.

### Example 4

### Hydrophilic Coating

20 g of polyvinyl-pyrrolidone (PVP K90) was mixed with 4 g of polyethylene-glycol dimethacrylat 1000 (PEG-DMA 1000) and 1 g natriumperoxidisulfate in 75 g of 0.1 M citric acid/citrate buffer pH 6.0. After the mixture had become homogeneous 0.30 g of anti-microbial polymer (AMINA® T 100 from Degusa) was dispersed. The polymer solution was dispensed into a suitable mold in 5 mm thickness and cured under UV-light. The hydrogel was UV-cured under a single UV-lamp (specifications: 200 W/cm, microwave powered “D”-spectral type lamp with a conveyor speed of 0.4 m/min). A sheet hydrogel of 5 mm thickness was obtained with anti-microbial properties.

### Example 5

### Hydrophilic Coating

A hydrophilic foam dressing was allowed to equilibrate in a vehicle solution (1% antimicrobial polymer solubilized water or ethanol) or various concentrations (1-0.008%) of the antimicrobial polymer solubilized in ethanol. The swollen foam was dried out by incubating the foam overnight at 40° C. Approximately 100 mg of foam was incubated for 1.5 hours in the presence of 5 ml of phosphate buffered saline containing 10⁷ CFU (colony forming units) per ml bacteria, and the collected samples were diluted and transferred to agar plates. The number of surviving bacteria was counted.

### Example 6

### Hydrogel

20 g of polyvinyl-pyrrolidone (PVP K90) was mixed with 4 g of polyethylene-glycol dimethacrylat 1000 (PEG-DMA 1000), 0.2 g of the antimicrobial polymer (AMINA® T 100 from Degusa) and 1 g natriumperoxidisulfate in 74.8 g of 0.1 M citric acid/citrate buffer pH 6.0. The polymer solution was dispensed into a suitable mold in 5 mm thickness and cured under UV-light. The hydrogel was UV-cured under a single UV-lamp (specifications: 200 W/cm, microwave powered “D”-spectral type lamp with a conveyor speed of 0.4 m/min). A sheet hydrogel of 5 mm thickness was obtained.

The equilibrium swelling was determined by swelling the cured hydrogels in Milli-Q water for 24 hours and calculating the relative increase in uptake of water. Despite the high amount of water, the hydrogel is still capable of absorbing water 7 times its own weight.

### Example 7

### Hydrogel

20 g of polyvinyl-pyrrolidone-co-vinylacetat (VA64) is mixed with

4 g of polyethylene-glycol dimethacrylat 1000 (PEG-DMA 1000), 0.2 g of the antimicrobial polymer (AMINA® T 100 from Degusa) and 1 g natriumperoxidisulfate in 59.8 g of 0.1 M citric acid/citrate buffer pH 6.0.

The polymer solution was dispensed into a suitable mold in 5 mm thickness and cured under UV-light. The hydrogel was UV-cured under a single UV-lamp (specifications: See Example 1). A sheet hydrogel of 5 mm thickness was obtained.

The equilibrium swelling was determined by swelling the cured hydrogels in Milli-Q water for 24 hours and calculating the relative increase in uptake of water as in example 1. The equilibrium swelling=850%.

### Example 8

### Hydrophilic Foam

### Materials:

100 parts w/w Hypol2002 (Dow Chemical Company)
1 part w/w Pluronic 62 (BASF)
100 parts w/w water
1.01*10⁻⁵-25.3 parts of (10⁻⁵ to 20 wt/vol %) of the anti-microbial polymer, a type that does not contain primary or secondary amines available for chemical reaction.

A polyurethane foam was prepared in the following way:

The materials are mixed together for approximately 15 seconds. The liquid is poured into a mould and allowed to react for 10 minutes. The resulting foam sheet is dried in an oven at 70° C. for 10 minutes.

### Further Handling Material:

The foam may be combined with other material e.g. film layers to constitute a device or part of a device. The material may further be sterilized using radiation.

### Example 9

### Hydrophilic Foam

### Materials Used for Recipe:

100 parts w/w Hypol2002 (Dow Chemical Company)
1 part w/w Pluronic 62 (BASF)
100 parts w/w water

A pre-synthesized foam based on following recipe is used for impregnation with an anti-microbial polymer.

The materials are mixed together for approximately 15 seconds. The liquid is poured into a mould and allowed to react for 10 minutes. The resulting foam sheet is dried in an oven at 70° C. for 10 minutes. The Foam is subsequently impregnated with solution of 1% anti-microbial polymer. The foam is dried at 40° C.

The foam may be combined with other material e.g. film layers to constitute a device or part of a device. The material may further be sterilized using radiation.

1. A hydrophilic, water-swellable, cross-linked matrix of a hydrophilic polymer having incorporated therein an anti-microbial polymer, said anti-microbial polymer carrying pendant groups providing the anti-microbial effect, said pendant groups being selected from primary amino groups, secondary amino groups, tertiary amino groups, quaternary ammonium groups, imino groups, and phosphonium groups.

2. The matrix according to claim 1, wherein the matrix is insoluble in water.

3. The matrix according to claim 1, wherein the hydrophilic matrix comprises at least one hydrophilic polymer selected from the group consisting of water-swellable hydrophilic polymers are those selected from polyvinylpyrrolidone, polyvinyl alcohol, polyacrylic acid, polymethacrylic acid, polyacrylic amides, polymethacryl amides, grafts or copolymers of any of these polymers, polyamides, polyethylene glycols (PEG), gelatine, polysaccharides, hydrophilic polyurethanes, and carboxylated butadiene styrene rubbers.

4. The matrix according to claim 1, the ratio of the thickness of the hydrophilic matrix in water swollen form (h_{water}) to the thickness of the hydrophilic matrix in dry form (h_{dry}) is at least 2:1.

5. The matrix according to claim 1, wherein the matrix is in the form of a hydrophilic coating on a substrate.

6. The matrix according to claim 1, wherein the matrix is in the form of a hydrogel.

7. The matrix according to claim 1, wherein the matrix is in the form of a hydrophilic foam.

8. An object comprising a substrate material and a layer covering at least a part of the surface of said substrate material, said layer consisting of a hydrophilic, water-swellable, cross-linked matrix as defined in claim 1.

9. The object according to claim 8, which is a medical device or a medical device element.

10. The object according to claim 9, wherein the medical device or medical device element is a catheter or catheter element, and the matrix is in the form of a hydrophilic coating.
